(19) United States
(12) Patent Application Publication
(10) Pub. No.: US 2004/0120994 A1
(43) Pub. Date: Jun. 24, 2004

(54) Transdermal therapeutic system containing testorone and method for its production thereof

(76) Inventor: Frank Theobald, Bad Breisig (DE)

Correspondence Address: BIRCH STEWART KOLASCH & BIRCH, PO BOX 747, FALLS CHURCH, VA, 22040-0747, US

(21) Appl. No.: 10/468,436

(22) PCT Filed: Feb. 7, 2002
(86) PCT No.: PCT/EP02/01258

(30) Foreign Application Priority Data
Feb. 19, 2001 (DE) 101 07 663.0

(51) Int. Cl. A61K 9/70

(52) U.S. Cl. 424/449

(57) ABSTRACT

A transdermal therapeutic system for administering sex hormones which is provided with an active substance-impermeable backing layer, a pressure-sensitive adhesive polymer matrix connected therewith and containing a sex hormone as well as skin penetration-enhancing substances, and with a protective layer detachable prior to application, is characterized in that said polymer matrix contains the sex hormone testosterone as well as a mixture of at least one penetration-enhancing substance from the group comprising fatty alcohol esters and fatty acid esters, and at least one readily volatile penetration-enhancing substance.

The invention relates to transdermal therapeutic systems (TTS) for administering sex hormones, which systems contain testosterone and a mixture of skin penetration-enhancing substances. The invention further relates to processes for the manufacture of such TTS.

Testosterone belongs to the group of sex hormones; it is the strongest natural androgen. The daily testosterone production amounts to about 7 mg (corresponding to 24 μmol) in men, and about 10% of that amount in women. In the blood, 98% of the testosterone is bound to transport proteins. The testosterone serum concentrations in men amount to 3 to 10 μg/l, corresponding to 10 to 35 nmol/l.

If the serum concentration of testosterone in men sinks below a value of 10 nmol/l, this is called the hypogonadism syndrome, which is characterized first of all by an incomplete formation or by a lack of formation, or a secondary involution of primary or secondary sex characters. The therapy of hypogonadism caused by testosterone deficiency consists in the substitution of testosterone.

Due to its short plasma half-life (around 80 min.) and intensive first-pass metabolism it is not possible to administer testosterone orally. As a rule testosterone is administered in the form of a suitable ester compound by intra-muscular injection.

On the other hand, due to its physicochemical properties, testosterone appears to be suitable for transdermal application. However, it is to be borne in mind that it should be possible to carry through the therapy in as inconspicuous and discrete a manner as possible, since hypogonadism is a disease that represents a heavy burden on the person concerned and may lead to social isolation or to the person withdrawing from his social environment. This is to be considered also when designing a transdermal therapeutic system, in order to ensure compliance and thereby the success of the therapy.

For example, transdermal therapeutic systems are known which are intended to be applied on the scrotum. This often necessitates pre-treatment of the scrotum by removing hair, which affects user friendliness and acceptance of such systems.

As an alternative, there are transdermal therapeutic systems which are conceived as reservoir systems. In such systems, testosterone is present dissolved in a solvent, for example in an alcohol. The release of testosterone to the skin is controlled by means of a control membrane. Such membrane-controlled systems have the advantage that they can be applied to the skin like other TTS known from the state of the art. They do have the disadvantage, however, that in the case of damage to the membrane so-called “dose dumping” may occur, i.e. the content of the active substance reservoir is delivered to the skin within a short period through the damaged membrane, which can lead to a preliminary over-dose. Furthermore, the solvents commonly used for the active substance reservoir, such as alcohols, in the high concentrations used in the reservoirs, frequently have a skin-irritating effect and cause reddening and itching at the site of application.

It was therefore the object of the present invention to provide a transdermal therapeutic system which enables the continuous delivery of testosterone to the skin and which does not have the above-described disadvantages.

This object is achieved by a transdermal therapeutic system (TTS) having the features mentioned in the preamble of claim 1 whose pressure-sensitive adhesive polymer matrix contains testorsterone and additionally a mixture of at least two substances enhancing skin penetration, namely at least one penetration-enhancing substance from the group comprising the fatty alcohol esters and fatty acid esters and at least one high-volatile penetration-enhancing substance. According to a preferred embodiment, both the hormone and the penetration-enhancing additives are homogenously distributed in the pressure-sensitive adhesive polymer matrix.

Within the framework of the studies on which this invention is based it has been found that certain mixtures of permeation enhancers (=skin penetration-enhancing substances) have an optimal penetration-enhancing effect for testosterone. These are mixtures of at least one fatty alcohol ester and/or fatty acid ester, and one or more high-volatile substance(s). Suitable as high-volatile enhancer substances are especially isopropylidene glycerol, transcutol (=diethylene glycol monoethyl ether), DEET (=N,N-diethyl-m-tolueneamide), solketal, ethanol, 1,2-propanediol, or other short-chain alcohols (i.e. alcohols with up to 6 C atoms), as well as menthol and other essential oils or components of essential oils.

As fatty alcohol ester, preferably ethyl oleate is used, or a fatty alcohol ester selected from the group of compounds comprising ethyl laurate, ethyl palmitate, ethyl lactate, propyl lactate, propyl palmitate, propyl laurate, propyl oleate, etc.

Utilized as fatty acid esters are preferably those selected from the compound group containing oleic acid ethyl ester, oleic acid methyl ester, lauric acid methyl ester, lauric acid ethyl ester, adipic acid methyl ester, adipic acid ethyl ester, etc.

Penetration-enhancing mixtures of the kind mentioned wherein the substance(s) from the group comprising fatty alcohol esters and fatty acid esters, and the readily volatile substance(s) is/are present in a relative quantitative ratio of from 1:2 to 2:1 have proved to be especially suitable. The amount of the readily volatile penetration-enhancing substance(s) amounts to preferably 10 to 20%-wt., with particular preference 15 to 20%-wt., each value relative to the active substance matrix. The amount of the penetration-enhancing substances from the group comprising fatty alcohol esters and fatty acid esters is preferably 5 to 20%-wt., especially preferred 6 to 10%-wt, each value relative to the matrix (i.e. without taking into account the nonwoven, the backing layer and the detachable protective layer).

It has furthermore turned out that adding nicotinic acid amide to the TTS according to the invention causes a further increase of the skin permeation rate. The concentration of the nicotinic acid amide here is preferably in the range of 2 to 10%-wt., with particular preference in the range of 3 to 5%-wt., each value relative to the active substance-containing matrix.

According to a particularly preferred embodiment, the testosterone-containing TTS according to the invention contain at least one penetration-enhancing substance from the group comprising fatty alcohol esters and fatty acid esters at a total concentration of from 5 to 20%-wt., preferably 6 to 10%-wt., as well as at least one substance selected from the group comprising isopropylidene glycerol, DEET, transcutol and short-chain alcohols at a total concentration of 10 to 20%-wt., preferably 15 to 20%-wt., and additionally nicotinic acid amide at a concentration of 2 to 10%-wt., preferably 3 to 5%-wt. The percentages indicated refer to the matrix.

The content of testosterone in the systems according to the invention is in the range of from 0.1 to 10%-wt., with particular preference in the range of from 1 to 5%-wt., each relative to the matrix. The term “testosterone” is understood to include testosterone esters as well. Possible testosterone esters are, in particular, testosterone acetate and testosterone propionate.

As pressure-sensitive adhesive matrix preferably a polymer layer produced on the basis of pressure-sensitive adhesive polymers from the group of polyacrylates is used with preference. Moreover, coatings produced on the basis of pressure-sensitive hot-melt adhesives may be used as pressure-sensitive adhesive matrix.

The pressure-sensitive adhesive polymer matrix may, apart from the polymer(s), the active substance and the enhancer substances, contain further auxiliaries known to those skilled in the art. Apart from that, the matrix is substantially comprised of pressure-sensitive adhesive polymers.

A further advantageous embodiment provides for the inventive testosterone-containing TTS to contain an antioxidant or an antioxidant combination, the portion of these substances preferably amounting to 0.1 to 5%-wt., with particular preference 0.3 to 1%-wt., each value being relative to the active substance-containing matrix. As antioxidant for testosterone-containing TTS, preferably tocopherol and ascorbyl palmitate are suitable.

On the side averted from the skin the active substance-containing polymer matrix is covered with an active substance-impermeable backing layer which is connected with said matrix.

Suitable as materials for the backing layer are first of all polyesters which stand out for their especially high strength such as, for example, polyethylene terephthalate and polybutylene terephthalate, but in addition almost any other skin-compatible plastics, such as polyvinyl chloride, ethylene-vinyl acetate copolymers, polyvinyl acetate, polyethylene, polypropylene, polyurethane, cellulose derivatives and many more. In the individual case the backing layer may be provided with an additional layer, e.g. by vapour deposition of metals, especially aluminium.

For the detachable protective layer, basically the same materials may be used as are used for the backing layer, provided that the protective layer is rendered detachable by a suitable surface treatment such as, for example, siliconization. Other detachable protective layers such as, for example polytetrafluoroethylene-treated paper or cellophane® (cellulose hydrate) may be used as well.

The manufacture of TTS having an active substance-containing matrix layer is usually carried out in such a manner that a solution or suspension of the active substance in an adhesive or non-adhesive polymer is prepared. This solution or suspension is coated, by means of a suitable coating unit, to a carrier material and subsequently the solvent present is removed by drying.

If the matrix systems to be produced, as in the present case, contain a readily volatile component, the abovedescribed approach is not possible, as otherwise the readily volatile component would evaporate. When the polymer matrix is prepared from the melt (hot-melt process), the same problems occur.

According to the invention, this problem is solved by applying the liquid mixture of enhancers, which optionally may contain in addition the active substance testosterone, in a defined amount onto a nonwoven fabric, a woven fabric (e.g. a textile fabric) or to a carrier film. This nonwoven fabric, woven fabric, or this carrier film is not subjected to drying. The thus pre-treated nonwoven or woven fabric, respectively the thus pre-treated carrier film is instead laminated onto an already previously prepared and dried polymer matrix layer. The nonwoven fabric or woven fabric is then connected with the matrix layer and preferably embedded therein, i.e. it has turned into a component of the matrix.

During the subsequent storage, diffusion occurs, resulting in a uniform, homogenous distribution of the active substance and the enhancer substances in the polymer matrix.

To the mixture of the penetration-enhancing substances, also designated as enhancer solution, may be added thickening agents and gelatinizing agents in order to adjust a viscosity that is suitable for carrying out the above-described process according to the present invention. Suitable for this are preferably substances from the group comprising polyacrylates, polyethylene glycol, polyvinyl pyrrolidone, polyvinyl alcohol, cellulose and cellulose derivatives.

A preferred embodiment of the production process according to the present invention therefore provides for the production of the inventive testosterone-containing TTS to be carried out in such a manner that first a polymer matrix is prepared by coating a solution of a pressure-sensitive adhesive polymer or polymer mixture to a film-shaped support and subsequent drying. In addition, a mixture of at least one penetration-enhancing substance from the group comprising fatty alcohol esters and fatty acid esters and at least one high-volatile penetration-enhancing substance is prepared. Subsequently, testosterone is added to the aforementioned mixture, dissolving testosterone in the mixture. The addition of testosterone may be omitted if the hormone has already been added to the solution of the pressure-sensitive adhesive matrix polymer.

The viscosity of this liquid enhancer mixture may optionally be adjusted in the above-described manner. Subsequently, the mixture containing the penetration-enhancing substances (and possibly testosterone) is applied to a nonwoven fabric or woven fabric or to a carrier film. This nonwoven fabric, woven fabric, or this carrier film, impregnated with enhancer mixture and possibly testosterone, is laminated to the dried polymer matrix so that it bonds with said matrix or is embedded therein. As a rule, the nonwoven fabric is located between two polymer layers (“sandwich”).

In the above-described production methods, testosterone may also be used in the form of its esters. As testosterone esters especially testosterone acetate and testosterone propionate are taken into consideration.

The above-mentioned backing layer or a film material suitable for the backing layer, as indicated above, may serve as the carrier film.

The nonwoven or woven fabric is preferably made of viscose, polyester, polypropylene, polyethylene, polyamide, cellulose, or of combinations of these materials.

The invention will be explained by way of the following examples, without, however, limiting the invention in any way.

### EXAMPLE 1

| Acrylate matrix: | |
|---|---|
| 1. Testosterone | 2.00% |
| 2. Durotak⁽¹⁾ | 90.70% |
| 3. Al-acetyl acetonate | 0.80% |
| 4. Nicotinic acid amide | 5.00% |
| 5. Tocopherol | 0.75% |
| 6. Ascorbyl palmitate | 0.75% |
| Thickened enhancer solution | |
| 1. Ethyl oleate | 21.70% |
| 2. Solketal | 43.40% |
| 3. Plastoid B⁽²⁾ | 27.90% |
| 4. Testosterone | 7.00% |

The acrylate matrix has a weight per unit area of 120 g/m². The thickened enhancer solution has a weight per unit area of 60 g/m². ⁽¹⁾Polyacrylate pressure-sensitive adhesive (by the firm of National Starch) ⁽²⁾Copolymerisate based on methacrylic acid and methacrylic acid methyl esters (manufacturer: Röhm GmbH)

### EXAMPLE 2

Further, a formulation having the following matrix layer composition proved to be especially suitable:

Testosterone . . . 3,5 Gew.-%

Nicotinic acid amide . . . 3,5 Gew.-%

Polyacrylate . . . 63,0 Gew.-%

Ethyl oleate . . . 10,0 Gew.-%

Isopropylidene glycerol . . . 20,0 Gew.-%

(The percentages relate to the pressure-sensitive adhesive polymer matrix).

The testosterone-containing TTS according to the invention may be advantageously employed in the substitution treatment of male hypogonadism.

Moreover, they are suitable for treating other testosterone deficiency-induced clinical pictures and symptoms, e.g. for the treatment of male climacteric symptoms (“hormone replacement therapy/HRT” for men), the treatment of male sterility, or of osteoporosis arising from androgen deficiency.

Making use of the anabolic effects imparted by testosterone, the TTS according to the invention may also be employed to give supporting treatment to HIV patients (AIDS) or tumour patients, and in addition to cases of other chronically consumptive diseases or states of disease involving catabolic metabolic conditions.

A further preferred area of indications of the testosterone-containing TTS according to the invention relates to the treatment of premenstrual syndrome (PMS) in women.

1. Transdermal therapeutic system for administering sex hormones which has an active substance-impermeable backing layer, a pressure-sensitive adhesive polymer matrix connected therewith and containing a sex hormone as well as skin penetration-enhancing substances, and a protective layer detachable prior to application, characterized in that said polymer matrix contains
the sex hormone testosterone as well as a mixture of
one or more penetration-enhancing substance(s) from the group comprising fatty alcohol esters and fatty acid esters, with particular preference ethyl oleate, in a total concentration of from 5 to 20%-wt., preferably 6 to 10%-wt, and
one or more readily volatile penetration-enhancing substance(s) from the group comprising isopropylidene glycerol, transcutol (=diethyleneglycolmonoethyl ether), DEET (=N,N-diethyl-m-tolueneamide), ethanol, 1,2-propanediol, short-chain alcohols, menthol, essential oils and components of essential oils, in a total concentration of from 10 to 20%-wt., preferably 15 to 20%-wt., and
nicotinic acid amide in a concentration of from 2 to 10%-wt., preferably 3 to 5%-wt.,
the concentrations indicated being relative to the weight of the matrix.

2. Transdermal therapeutic system according to claim 1, characterized in that the substance (s) from the group comprising fatty alcohol esters and fatty acid esters, on the one hand, and the substance(s) from the group of the readily volatile substances, on the other hand, are present in the said mixture in a relative quantitative ratio of from 1:2 to 2:1.

3. Transdermal therapeutic system according to claim 1 or 2, characterized in that the polymer matrix is a matrix based on polyacrylates.

4. Transdermal therapeutic system according to claim 1 or 2, characterized in that the polymer matrix is a matrix based on pressure-sensitive hot-melt adhesives.

5. Transdermal therapeutic system according to any one of claims 1 to 4, characterized in that the polymer matrix has a nonwoven fabric or a woven fabric or a carrier film which is/are impregnated with the penetration-enhancing substances mentioned, or with the penetration-enhancing substances mentioned and testosterone, the nonwoven or woven fabric or the carrier film being connected with the polymer matrix, preferably being embedded in the polymer matrix.

6. Transdermal therapeutic system according to any one of claims 1 to 5, characterized in that testosterone is present as ester, preferably as testosterone acetate or testosterone propionate.

7. Transdermal therapeutic system according to any one of claims 1 to 6 characterized in that the testosterone content amounts to 1 to 10%-wt., preferably 1 to 5%-wt., relative to the matrix.

8. Transdermal therapeutic system according to any one of claims 1 to 7, characterized in that it contains an antioxidant or a combination of antioxidants, preferably a combination of tocopherol and ascorbyl palmitate, the content of the antioxidant/antioxidants being 0.1 to 5%-wt., preferably 0.3 to 1%-wt., each value relative to the matrix.

9. Transdermal therapeutic system according to any one of claims 1 to 8, characterized in that it has a portion of additives from the group of the thickening agents and gelatinizing agents, preferably selected from the group comprising polyacrylates, polyethylene glycol, polyvinyl pyrrolidone, polyvinyl alcohol, cellulose and cellulose derivatives.

10. Transdermal therapeutic system according to any one of claims 1 to 9, characterized in that the active substance and the penetration-enhancing substances are completely dissolved and homogenously distributed in the system.

11. Process for producing a testosterbne- and penetration-enhancing additives-containing transdermal therapeutic system, characterized in that
by coating a solution or melt of a pressure-sensitive adhesive polymer or of a polymer mixture to a film-shaped support and subsequent drying, a polymer matrix is prepared;
a mixture of at least one penetration-enhancing substance from the group comprising fatty alcohol esters and fatty acid esters and at least one readily volatile penetration-enhancing substance is prepared;
testosterone is added to the afore-mentioned mixture, dissolving the said testosterone in the mixture;
the mixture containing testosterone and penetration-enhancing substances is applied to a nonwoven fabric or woven fabric or to a carrier film;
this nonwoven fabric, woven fabric or carrier film is laminated to the dried polymer matrix.

12. Process for producing a testosterone- and penetration-enhancing additives-containing transdermal therapeutic system, characterized in that
by coating a testosterone-containing solution or melt of a pressure-sensitive adhesive polymer or polymer mixture to a film-shaped support and subsequent drying, a polymer matrix is prepared;
a mixture of at least one penetration-enhancing substance from the group comprising fatty alcohol esters and fatty acid esters and at least one readily volatile penetration-enhancing substance is prepared;
the penetration enhancing substances-containing mixture is applied to a nonwoven or woven fabric or a carrier film;
this nonwoven fabric, woven fabric or carrier film is laminated to the dried polymer matrix.

13. Process according to claim 11 or 12, characterized in that to the liquid penetration-enhancing mixture is added at least one component for adjusting the viscosity, said component preferably being selected from the group of thickening agents and gelatinizing agents, with particular preference from the group comprising polyacrylates, polyethylene glycol, polyvinyl pyrrolidone, polyvinyl alcohol, cellulose and cellulose derivatives.

14. The use of a transdermal therapeutic system according to any one of claims 1 to 10 for hormone substitution in the treatment of male hypogonadism, or for hormone substitution in cases of testosterone deficiency in men caused by old age, or as anabolic agent in the treatment of HIV and carcinoses, or for the treatment of premenstrual syndrome in women.
